(19) United States
(12) Patent Application Publication
(10) Pub. No.: US 2022/0225653 A1
(43) Pub. Date: Jul. 21, 2022

(54) METHODS FOR THE PRODUCTION OF MYCELIATED BULKING COMPOSITIONS

(71) Applicant: MycoTechnology, Inc., Aurora, CO (US)

(72) Inventors: Bhupendra Kumar SONI, Aurora, CO (US); Brendan SHARKEY, Aurora, CO (US); Alan D. HAHN, Aurora, CO (US); James Patrick LANGAN, Aurora, CO (US); Brooks John KELLY, Aurora, CO (US); Anthony J. CLARK, Aurora, CO (US)

(73) Assignee: MycoTechnology, Inc., Aurora, CO (US)

(21) Appl. No.: 17/608,581

(22) PCT Filed: May 8, 2020
(86) PCT No.: PCT/US20/32065

§ 371 Date: Nov. 3, 2021

Related U.S. Application Data
(60) Provisional application No. 62/845,128, filed on May 8, 2019.
(60) Provisional application No. 62/886,249, filed on Aug. 13, 2019.
(60) Provisional application No. 62/888,031, filed on Aug. 16, 2019.

(51) Int. Cl. A23L 31/00; A01G 18/20

(52) U.S. Cl. CPC A23L 31/00; A23P 30/20; A01G 18/20

(57) ABSTRACT

Disclosed is a method to prepare a myceliated low-quality protein composition, which includes culturing a filamentous fungus an aqueous media. Examples of low-quality protein compositions include corn gluten meal. After culturing, the material is harvested by obtaining the myceliated low-quality protein composition via drying or concentrating. The resultant composition may have its taste, flavor, or aroma modulated, such as by deflavoring and/or deodorizing. Also disclosed are myceliated low-quality protein compositions, food products comprising such compositions, and methods to make such products.

### CROSS-REFERENCE TO RELATED APPLICATIONS

This application claims the benefit of priority to U.S. Provisional Patent Application No. 62/845,128, filed May 8, 2019; U.S. Provisional Patent Application No. 62/886,249, filed Aug. 13, 2019; and U.S. Provisional Patent Application No. 62/888,031 Aug. 16, 2019, all of which are specifically incorporated by reference to the extent not inconsistent herewith.

### BACKGROUND OF THE INVENTION

Bulking agents are primarily useful as fillers and can provide inexpensive extenders for costly ingredients such as cocoa butter or nonfat dry milk. Bulking agents can be used in food products such as spreads, pastes, prewhipped toppings, custards, coatings, nut butters, frostings, cream filings, confectionery fillings, to decrease costs and optionally, increase nutrition of the food product. Bulking agents can also replace or partially replace ingredients such as sugar and/or fat to lower calories and sugar from food products and/or increase nutrients (such as proteins). Bulking agents should be relatively bland in taste and provide functional attributes.

Fiber can provide bulk, as well as a health-oriented image. Fiber ingredients come from a number of sources and typically contain a mixture of soluble and insoluble fiber. Most fiber ingredients, especially insoluble forms, are in the form of flours derived from plants—grains like wheat, soy and oats; legumes; fruit. Fiber ingredients comprising cellulose is considered a bulking grade. Those would have a very fine consistency and physically resemble flour. They perform very well as bulking agents for sugar and fat, as well as for flour.

One such bulking agent is a flour derived from a legume or other non-wheat material. Examples of legume starches are pea starches, such as wrinkled pea or smooth pea starch, fava bean, mung bean, red kidney bean, and lentil bean starch. Another starch-containing material is chicory root (e.g., powder). The root contains up to 20% inulin, a polysaccharide similar to starch. Inulin is also gaining popularity as a source of soluble dietary fiber and functional food. Fresh chicory root typically contains, by dry weight, 68% inulin, 14% sucrose, 5% cellulose, 6% protein, 4% ash, and 3% other compounds. Dried chicory root extract contains, by weight, about 98% inulin and 2% other compounds. Fresh chicory root may contain between 13 and 23% inulin, by total weight. However, it has a bitter taste which is believed to be due to sesquiterpene lactones and other ingredients. Grapeseed (e.g., powder) is another starch-containing material. Generally, this flour is made from the seeds of grapes after oil is removed from the seeds by cold pressing. Beer grains (e.g., powder) is a product comprising different mixtures of various malted grains, including barley, corn, oats, rice, rye, and/or wheat. These materials may also contain a bitter or other off-note.

However, fiber-containing bulking agents can be low in nutrients, such as protein. There are a number of plant proteins that have the potential to support global protein production by partially replacing meat and dairy products in the human diet. However, many of these plant proteins have off-flavors such as unpleasant tastes and aromas. For example, fava bean contain compounds to cause off-flavors, including anthocyanidins which have been shown to activate bitter taste receptors, while vanillic, caffeic, p-coumaric, and ferulic acids ethyl esters have a bitter character, in addition to an astringent character. Regular fava beans contain up to 8-9% tannins, which can explain their perceived bitterness. Corn gluten meal is another protein. Corn protein, unlike soy, is not a major allergen. Corn gluten meal typically contains about 65% crude protein, and is typically used only for livestock feed for the primary reason that corn gluten meal has undesirable sensory characteristics. Its unpleasant taste and odor have limited its ability to be used as a human food. Corn gluten meal appears yellow in color due to the presence of xanthophylls, which is also an undesired characteristic in a protein for human consumption.

However, there remains a need for a way to utilize low cost, but organoleptically undesirable, bulking materials in human foods, and additionally, find low-cost ways to increase nutrients such as protein using low-cost protein sources. These protein sources also have organoleptic challenges. However, it has proven difficult to achieve such products.

### SUMMARY OF THE INVENTION

In an embodiment, the present invention includes a method to prepare an improved composition comprising at least one low quality protein, which can include the following steps. In one step, provided is an aqueous media comprising at least one low quality protein, wherein the media comprises at least 10 g/L low quality protein; in another step, the media is inoculated with a filamentous fungal culture, wherein the fungal culture comprises, consists of, or consists essentially of, Lentinula spp., Pleurotus spp., or Morchella spp. The method further comprises culturing the medium in submerged fungal culture to produce a myceliated low quality protein composition. The myceliated low quality protein composition has improved aroma and/or improved taste and/or decreased bitter taste, compared to a low-quality protein composition that is not myceliated. The myceliated low quality protein composition can be used as a protein composition or a bulking composition. It may have decreased beany, bitter or earthy flavors and/or decreased beany, earthy or sulfur aromas compared to a control. Low quality protein includes, in some embodiments, fava bean protein and corn gluten meal. Additional high protein materials to include in the media include pea protein and/or chickpea. In embodiments, the filamentous fungus is selected from the group of L. edodes, M esculenta, and P. salmoneostramineus.

The invention also includes compositions made by the methods of the invention. In one embodiment, the composition comprises a myceliated low quality protein composition, wherein the myceliated low quality protein composition is at least 20% (w/w) protein on a dry weight basis, wherein myceliated low quality protein composition is myceliated by filamentous fungal culture comprising, consisting of, or consisting essentially of Lentinula spp., Pleurotus spp., and/or Morchella spp. in a media comprising at least 20 g/L protein, and wherein the myceliated low quality protein composition has improved aroma and/or improved taste and/or decreased bitter taste, compared to a low quality protein composition that is not myceliated.

The present invention also includes food compositions comprising the myceliated low quality protein compositions, and includes food compositions such as spreads, pastes such as sweet (e.g. chocolate or fruit) pastes or savory pastes, prewhipped toppings, custards, coatings, peanut butter, frostings, cream filings, confectionery fillings, dairy alternative products, beverages and beverage bases, extruded and extruded/puffed products, meat imitations and extenders, baked goods and baking mixes, granola products, bar products, smoothies and juices, and soups and soup bases.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

The present inventors have found that culturing a filamentous fungus in an aqueous media that includes one or more low-quality proteins (or “ingredients”) to make a myceliated low-quality protein composition comprising at least one low-quality protein, provides an economically viable product, and also found that such treatment can also alter the taste, flavor, aroma, color of one or more one or more low-quality protein-containing food compositions in unexpected ways. The process additionally enables the production of a bulking ingredient with improved nutrition, e.g., improved protein content, having improved taste, flavor, aroma, and/or color, that has been imbued with mycelial material.

In an embodiment, the inventors have achieved a vegetarian, vegan source of bulking ingredients and/or protein composition, that uses proteins not normally not used for human consumption due to poor PDCAAS, or due to flavor and taste (sensory) defects, as well as anti-nutritive factors, and/or undesirable color. In an embodiment, the protein originates only from plant-based sources, and after myceliation according to the present invention, has a flavor profile that includes, for example, reduced undesirable aromas, reduced undesirable flavors, change in color, increased solubility, and reduced antinutritive factors. In an embodiment, it is possible to achieve a PDCAAS score that improves on the low-quality protein when combined with at least one other source of plant protein. For example, approximately 30% of corn gluten meal can be combined with about 70% of pea protein to yield a material with a PDCAAS of approximately 0.94. In an embodiment, the low-quality protein or bulking composition is rendered more acceptable for human (or animal) consumption by the processes of the present invention.

In one embodiment, the present invention includes a method to prepare a myceliated e.g., cultured, e.g., improved, composition comprising at least one low-quality protein (e.g., a food product). The method may optionally include the steps of providing an aqueous media comprising a comprising at least one low-quality protein, wherein the media comprises at least 10 g/L low-quality protein. The aqueous media may optionally, comprise, consist of, or consist essentially of at least 50% protein, on a dry weight basis; or may comprise, consist of, or consist essentially of at least 20% protein on a dry weight basis. The media may be in the form of a slurry with incomplete solubilization of the one or more of the components. The media may also comprise, consist of or consist essentially of optional additional components, excipients and other materials as identified herein below. The aqueous media may be inoculated with a filamentous fungal culture. The inoculated media may then be cultured to produce a myceliated one or more improved compositions, and the myceliated improved compositions may improve taste, flavor, aroma, color, solubility, among others, as compared to the composition in the absence of the culturing step.

In embodiments, mixtures of low-quality proteins, high quality proteins, and other, bulking ingredients may be used to form either a bulking composition with improved nutrition and/or a protein composition for use in typical protein applications, as described hereinbelow.

The aqueous media may comprise, consist of, or consist essentially of a low-quality protein together with a bulking ingredient. A bulking ingredient may be any bulking ingredient known in the art, and may include cereals, which can optionally include the seed coat and germ, a dry powdered whole grain powder. Bulking ingredients include cereals, brown rice, germinated brown rice, barley, wheat, oats, pearl barley, sorghum, buckwheat, millet, sesame, millet, millet, amaranth, quinoa, corn. In embodiments, the cereals include spent beer grains and/or rice bran fiber. In embodiments, the bulking ingredient includes a flour including amaranth flour, arrow root flour, buckwheat flour, rice flour, chickpea flour, cornmeal, maize flour, millet flour, potato flour, potato starch flour, quinoa flour, sorghum flour, soy flour, bean flour, legume flour, tapioca (cassava) flour, teff flour, artichoke flour, almond flour, a corn flour, coconut flour, chestnut flour, corn flour and taro flour. Bulking ingredients include roots, such as, without limitation, carrot, radish, turnip, potato, sweet potato, chicory, yam, taro, taro, konjak, burdock, ginseng, lotus root, turmeric, Udo, bean sprouts. Bulking ingredients also include seeds, such as, for example, grapeseed.

A “low-quality protein,” includes vegetable proteins which typically have lower PDCAAS scores than meats, and can include proteins with PDCAAS scores below 60, for example, indicating a deficiency of one or more essential amino acids, typically low in lysine (corn) or low in tryptophan (beans). In embodiments, a “low-quality protein” also includes proteins, that, in embodiments, refer to plant proteins that is typically not suitable for human ingestion due to such factors as organoleptic challenges including undesirable flavors, aromas and/or tastes. Low-quality proteins, in this definition, typically exclude plant proteins that are used for human consumption in significant amounts, such as pea protein, soybean, oat protein, hemp protein, chickpea flour, chia powder, cyanobacteria or algal protein, and the like. Such low-quality proteins include protein isolates and concentrates (or whole unprocessed, optionally milled) from proteins such as fava bean protein, red beans, broad beans, sunflower meal, canola meal, DDGS meal, copra meal, lupin meal, lemna meal, and the like; and in particular, corn gluten meal.

Vegetarian sources of additional sources of high protein material to optionally include with the low-quality protein materials in the aqueous media include meal, protein concentrates and isolates prepared from a vegetarian source such as pea, rice, soy, cyanobacteria, grain, hemp, chia, chickpea, potato protein, algal protein, oat, cyanobacteria containing more than 50% protein, nettle protein or combinations or subcombinations of these. In one embodiment, the protein is derived from a pulse (seed) from a legume, such as pea, chickpea, lentils, lupins, common beans (kidney, nay, pinto). In embodiments, the additional vegetarian source are protein(s) prepared from pea, rice, chickpea or a combination thereof. In embodiments, the additional vegetarian source are protein(s) prepared from pea, chickpea or a combination thereof. In one embodiment, the media may comprise pea protein, chickpea, and corn gluten meal.

Typically, a protein concentrate is made by removing the oil and retaining the meal, or may be a whole ingredient. The bulking ingredient may still contain a majority of non-protein material, such as fiber. Typically, protein concentrations in such products are between 25-90%. The process for production of a protein isolate typically removes most of the non-protein material such as fiber and may contain up to about 90-99% protein. A typical protein isolate is typically subsequently dried and is available in a powdered form and may alternatively be called “protein powder.”

The bulking ingredient may be used in an intact state, or may be partially or completely ground into, for example, a flour. In embodiments, a flour-like state is useful for facilitating additional surface area for facilitating fermentation.

In one embodiment, mixtures of any of the one or more proteins disclosed can be used to provide, for example, favorable qualities, such as a more complete (in terms of amino acid composition) low-quality protein composition. In other embodiments, low-quality protein compositions may be supplemented by using amino acids in purified or partially purified form. In one embodiment, low-quality protein compositions can be combined with protein materials from legume sources, such as pea protein. In one embodiment, the ratio can include mixtures that are 10-60% pea protein; 10-60% corn gluten meal; and/or 10-60% chickpea flour.

The protein material to add to the media, itself can be unprocessed (or, optionally milled) or a concentrate or isolate of at least about 20% protein, 30% protein, 40% protein, 45% protein, 50% protein, 55% protein, 60% protein, 65% protein, 70% protein, 75% protein, 80% protein, 85% protein, 90% protein, 95% protein, or 98% protein, or at least about 20% protein, at least about 30% protein, at least about 40% protein, at least about 45% protein, at least about 50% protein, at least about 55% protein, at least about 60% protein, at least about 65% protein, at least about 70% protein, at least about 75% protein, at least about 80% protein, at least about 85% protein, at least about 90% protein, at least about 95% protein, or at least about 98% protein.

This invention discloses the use of concentrated media, which provides, for example, an economically viable economic process for production of an acceptably tasting and/or flavored one or more low-quality protein composition. In one embodiment of the invention the total media concentration is up to 150 g/L but can also be performed at lower levels, such as 5 g/L. Higher concentrations in media result in a thicker and/or more viscous media, and/or media at least partially comprised of a slurry, and therefore are optionally processed by methods known in the art to avoid engineering issues during culturing or fermentation. In an embodiment, for efficiency, a greater amount of one or more ingredients per liter of media is used. The amount is used is chosen to maximize the amount of one or more ingredients that is cultured, while minimizing technical difficulties in processing that may arise during culturing such as viscosity, foaming and the like. The amount to use can be determined by one of skill in the art, and will vary depending on the method of fermentation.

In another embodiment, the aqueous media comprises between about 1 g/L and 200 g/L, between about 5 g/L and 180 g/L, between about 20 g/L and 150 g/L, between about 25 g/L and about 140 g/L, between about 30 g/L and about 130 g/L, between about 35 g/L and about 120 g/L, between about 40 g/L and about 110 g/L, between about 45 g/L and about 105 g/L, between about 50 g/L and about 100 g/L, between about 55 g/L and about 90 g/L, or about 75 g/L ingredient; or between about 50 g/L-150 g/L, or about 75 g/L and about 120 g/L, or about 85 g/L and about 100 g/L of one or more low-quality protein or total media components. Alternatively, the aqueous media comprises at least about 10 g/L, at least about 15 g/L, at least about 20 g/L, at least about 25 g/L, at least about 30 g/L, at least about 35 g/L, at least about 40 g/L or at least about 45 g/L of one or more low-quality protein or total media components.

In some embodiments, the aqueous media comprises between about 50 g/L and about 100 g/L, or about 80 g/L, about 85 g/L, about 90 g/L, about 95 g/L about 100 g/L, about 110 g/L, about 120 g/L, about 130 g/L, about 140 g/L, or about 150 g/L of one or more low-quality protein or total media components.

In some embodiments, the media may be partially dissolved, and/or partially suspended, and/or partially colloidal. However, even in the absence of complete dissolution of, positive changes may be affected during culturing. In one embodiment, the ingredients in the aqueous media are kept as homogenous as possible during culturing, such as by ensuring agitation and/or shaking.

In one embodiment, the aqueous media further comprises, consists of, or consists essentially of excipients as defined herein and/or in particular embodiments. Excipients can comprise any other components known in the art to potentiate and/or support fungal growth, and/or aid in processing (processing aids), and can include, for example, nutrients, such as proteins/peptides, amino acids as known in the art and extracts, such as malt extracts, meat broths, peptones, yeast extracts and the like; energy sources known in the art, such as carbohydrates; essential metals and minerals as known in the art, which includes, for example, calcium, magnesium, iron, trace metals, phosphates, sulphates; anti-foam agents; buffering agents as known in the art, such as phosphates, acetates, and optionally pH indicators (phenol red, for example). In one embodiment, the aqueous media contains antifoam excipients (processing aids) only.

In embodiments, the aqueous media further comprises, consists of, or consists essentially of at least one exogenously-added additional amino acid, purified or partially purified. The amino acid may be used to supplement the low-quality protein material where its amino acids are low, e.g., to create a material with a better PDCAAS score by adding, e.g., lysine, sulfur amino acids, and/or tryptophan. In an embodiment, the at least one amino acid may be at least one branched chain amino acid (“BCAA”) which is exogenously added to the high-protein material to increase the BCAA content. Examples of sources include Ajinomoto AMINO L40, which contains 9 essential amino acids (L-leucine, L-lysine, L-valine, L-isoleucine, L-threonine, L-phenylalanine, L-methionine, L-histidine, L-tryptophan).

Excipients may also include peptones/proteins/peptides, as is known in the art to support fungal growth. These are usually added as a mixture of protein hydrolysate (peptone) and meat infusion. Many media have, for example, between 1% and 5% peptone content, and between 0.1 and 5% yeast extract and the like.

In one embodiment, excipients include for example, yeast extract, malt extract, maltodextrin, peptones, and salts such as diammonium phosphate and magnesium sulfate, as well as other defined and undefined components such as potato or carrot powder. In some embodiments, organic (as determined according to the specification put forth by the National Organic Program as penned by the USDA) forms of these components may be used.

In one embodiment, excipients comprise, consist of, or consist essentially of dry carrot powder, dry malt extract, diammonium phosphate, magnesium sulfate, and citric acid. In one embodiment, excipients comprise, consist of, or consist essentially of dry carrot powder between 0.1-10 g/L, dry malt extract between 0.1 and 20 g/L, diammonium phosphate between 0.1 and 10 g/L, and magnesium sulfate between 0.1 and 10 g/L. Excipients may also optionally comprise, consist of, or consist essentially of citric acid and an anti-foam component.

1 The method may also comprise the optional step of sterilizing the aqueous media prior to inoculation by methods known in the art, including steam sterilization and all other known methods to allow for sterile procedure to be followed throughout the inoculation and culturing steps to enable culturing and myceliation by pure fungal strains. Alternatively, the components of the media may be separately sterilized and the media may be prepared according to sterile procedure.

Applicants have filed U.S. Pat. No. 10,010,103, filed Apr. 14, 2017, U.S. Ser. No. 16/025,365, (filed Jul. 2, 2018), both entitled “Methods for the Production and use of Myceliated High Protein Food Compositions,”, U.S. Ser. No. 62/752,158 (filed Oct. 29, 2018), U.S. Ser. No. 62/796,438 (filed Jan. 24, 2019), related to aqueous-phase fermentation of protein materials, all of which are incorporated by reference herein in their entireties. Inoculating the aqueous media with a filamentous fungal culture, wherein the filamentous fungal culture can include, comprise, consist of, or consist essentially of Lentinula spp., Pleurotus spp., or Morchella spp, and culturing the medium to produce a myceliated low-quality protein composition.

The filamentous fungal cultures, prior to the inoculation step, may be propagated and maintained as is known in the art.

In one embodiment, maintaining and propagating fungi for use for inoculating the one or more ingredients as disclosed in the present invention may be carried out as known in the art.

In some embodiments, liquid cultures used to maintain and propagate fungi for use for inoculating the one or more ingredients as disclosed in the present invention include undefined agricultural media with optional supplements as a motif to prepare culture for the purposes of inoculating solid-state material or larger volumes of liquid. In some embodiments, liquid media preparations are made as disclosed herein. Liquid media can be also sterilized and cooled similarly to agar media. Bioreactors provide the ability to monitor and control aeration, foam, temperature, and pH and other parameters of the culture and as such enables shorter myceliation times and the opportunity to make more concentrated media.

In one embodiment, the filamentous fungi for use for inoculating the one or more ingredients disclosed in the present invention may be prepared as a submerged liquid culture and agitated on a shaker table, or may be prepared in a shaker flask, by methods known in the art and according to media recipes disclosed in the present invention. The fungal component for use in inoculating the aqueous media of the present invention may be made by any method known in the art. In one embodiment, the fungal component may be prepared from a glycerol stock, by a simple propagation motif of Petri plate culture to 0.5 to 4 L Erlenmeyer shake flask to 50% glycerol stock. Petri plates can comprise agar in 10 to 35 g/L in addition to various media components. Conducted in sterile operation, chosen Petri plates can be propagated into 0.5 to 4 L Erlenmeyer flasks (or 250 to 1,000 mL Wheaton jars, or any suitable glassware) for incubation on a shaker table or stationary incubation. In one embodiment, the shaking is anywhere from 40-160 RPM depending on container size and, with about a 1″ swing radius.

The culturing step of the present invention may be performed by methods (such as sterile procedure) known in the art and disclosed herein and may be carried out in a fermenter, shake flask, bioreactor, or other methods. In an embodiment the incubation temperature is 70-90° F. Liquid-state fermentation agitation and swirling techniques as known in the art are also employed which include mechanical shearing using magnetic stir bars, stainless steel impellers, injection of sterile high-pressure air, the use of shaker tables and other methods such as lighting regimen, batch feeding or chemostatic culturing, as known in the art.

In one embodiment, culturing step is carried out in a bioreactor which is ideally constructed with a torispherical dome, cylindrical body, and spherical cap base, jacketed about the body, equipped with a magnetic drive mixer, and ports to provide access for equipment comprising DO, pH, temperature, level and conductivity meters as is known in the art. Any vessel capable of executing the methods of the present invention may be used. In another embodiment the set-up provides 0.1-5.0 ACH. Other engineering schemes known to those skilled in the art may also be used.

The reactor can be outfitted to be filled with water. The water supply system is ideally water for injection (WFI) system, with a sterilizable line between the still and the reactor, though RO or any potable water source may be used so long as the water is sterile. In one embodiment the entire media is sterilized in situ while in another embodiment concentrated media is sterilized and diluted into a vessel filled water that was filter and/or heat sterilized, or sufficiently treated so that it doesn't encourage contamination over the colonizing fungus. In another embodiment, high temperature high pressure sterilizations are fast enough to be not detrimental to the media. In one embodiment the entire media is sterilized in continuous mode by applying high temperature between 1300 and 150° C. for a residence time of 1 to 15 minutes. Once prepared with a working volume of sterile media, the tank can be mildly agitated and inoculated. Either as a concentrate or whole media volume in situ, the media can be heat sterilized by steaming either the jacket, chamber or both while the media is optionally agitated. The medium may optionally be pasteurized instead.

In one embodiment, the reactor is used at a large volume, such as in 500,000-200,000 L working volume bioreactors. When preparing material at such volumes the culture must pass through a successive series of larger bioreactors, any bioreactor being inoculated at 0.5-15% of the working volume according to the parameters of the seed train. A typical process would pass a culture from master culture, to Petri plates, to flasks, to seed bioreactors to the final main bioreactor when scaling the method of the present invention. To reach large volumes, 3-4 seeds may be used. The media of the seed can be the same or different as the media in the main. In one embodiment, the fungal culture for the seed is a one or more ingredients as defined herein, to assist the fungal culture in adapting to one or more ingredients media in preparation for the main fermentation. In one embodiment, foaming is minimized by use of antifoam on the order of 0.5 to 2.5 g/L of media, such as those known in the art, including insoluble oils, polydimethylsiloxanes and other silicones, certain alcohols, stearates and glycols. In one embodiment, lowering pH assists in culture growth, for example, for L. edodes pH may be adjusted by use of citric acid or by any other compound known in the art, but care must be taken to avoid a sour taste for the myceliated one or more ingredients. The pH may be adjusted to between about 4.5 and 5.5, for example, to assist in growth.

In one embodiment, during the myceliation step, for example, the pH does not change during processing. “pH does not change during processing” is understood to mean that the pH does not change in any significant way, taking into account variations in measured pH which are due to instrument variations and/or error. Such lack of change may indicate lack of mycelial growth, for example, if the mycelia enter lag phase upon change of media in the final fermentation. For example, the pH will stay within about plus or minus 0.3 pH units, plus or minus 0.25 pH units, plus or minus 0.2 pH units, plus or minus 0.15 pH units, or plus or minus 0.1 pH units of a starting pH of the culture during the myceliation, e.g. processing step.

In one embodiment, a preparation of L. edodes, P. salmoneostramineus, and/or M. esculenta as the filamentous fungal component for use for inoculating an aqueous media was prepared and used to create the myceliated one or more low-quality protein composition food product. In this embodiment, a one low-quality protein composition containing media was prepared and inoculated with L. edodes, P. salmoneostramineus, and/or M. esculenta. The increase in biomass concentration was correlated with a drop in pH. After shaking for 1 to 10 days, an aliquot (e.g. 10 to 500 mL) of the shake flask may be transferred in using sterile procedure into a sterile, prepared sealed container (such as a customized stainless steel can or appropriate conical tube), which can then adjusted with about 5-60%, sterile, room temperature (v/v) glycerol. The glycerol stocks may be sealed with a watertight seal and can be held stored at −20° C. for storage. The freezer is ideally a constant temperature freezer. Glycerol stocks stored at 4° C. may also be used. Agar cultures can be used as inoculant for the methods of the present invention, as can any culture propagation technique known in the art.

It was found that not all fungi are capable of growing in media as described herein. Fungi useful for the present invention are from the higher order Basidio- and Ascomycetes, e.g., filamentous fungi. In some embodiments, filamentous fungi effective for use in the present invention include (e.g., comprise, consist of, or consist essentially of), but are not limited to, Lentinula spp., such as L. edodes, Agaricus spp., such as A. blazei, A. bisporus, A. campestris, A. subrufescens, A. brasiliensis, or A. silvaticus; Pleurotus spp., Boletus spp., Morchella spp. or Laetiporus spp. In one embodiment, the fungi for the invention include fungi from optionally, liquid culture of species generally known as oyster, porcini, ‘chicken of the woods’ and shiitake mushrooms. These include Morchella spp. (morel). Morchella spp. can include, without limitation, all species of genus Morchella.

In embodiments, additional Morchella species suitable for the invention can optionally include Morchella angusticeps, Morchella importuna, Morchella americana, Morchella castaneae, Morchella diminutiva Morchella dunensis, Morchella fluvialis, Morchella galilaea, Morchella palazonii, Morchella prava, Morchella sceptriformis, Morchella steppicola, Morchella ulmaria, Morchella vulgaris, Morchella angusticeps, Morchella arbutiphila, Morchella australiana, Morchella brunnea, Morchella conifericola, Morchella deliciosa, Morchella disparilis, Morchella dunalii, Morchella elata, Morchella eohespera, Morchella eximia, Morchella eximioides, Morchella exuberans, Morchella feekensis, Morchella importuna, Morchella kakiicolor, Morchella laurentiana, Morchella magnispora, Morchella mediteterraneensis, Morchella populiphila, Morchella pulchella, Morchella punctipes, Morchella purpurascens, Morchella semilibera, Morchella septentrionalis, Morchella sextelata, Morchella snyderi, Morchella tomentosa, Morchella tridentina, Morchella anteridiformis, Morchella apicata, Morchella bicostata, Morchella conicopapyracea, Morchella crassipes, Morchella deqinensis, Morchella distans, Morchella guatemalensis, Morchella herediana, Morchella hetieri, Morchella hortensis, Morchella hotsonii, Morchella hungarica, Morchella inamoena, Morchella intermedia, Morchella meiliensis, Morchella miyabeana, Morchella neuwirthii, Morchella norvegiensis, Morchella patagonica, Morchella patula, Morchella pragensis, Morchella procera, Morchella pseudovulgaris, Morchella rielana, Morchella rigida, Morchella rigidoides, Morchella smithiana, Morchella sulcate, Morchella tasmanica, Morchella tatari, Morchella tibetica, Morchella umbrina, Morchella umbrinovelutipes, or Morchella vaporaria.

In a particular embodiment, the Morchella spp. consists of, consists essentially of, or comprises Morchella esculenta. Fungi for use in the present invention also include Pleurotus (oyster) species such as Pleurotus ostreatus, Pleurotus salmoneostramineus (Pleurotus djamor), Pleurotus eryngii, or Pleurotus citrinopileatus; Boletus (porcini) species such as Boletus edulis; Laetiporus (chicken of the woods) species such as Laetiporus sulfureus, and many others such as L. budonii, L. miniatus, L. flos-musae, L. discolor; and Lentinula (shiitake) species such as L. edodes. Also included are Lepista nuda, Hericium erinaceus, Agaricus blazeii, and combinations thereof. In one embodiment, the fungi is L. edodes, P. salmoneostramineus, and/or M. esculenta. Fungi may be obtained commercially, for example, from the Penn State Mushroom Culture Collection.

Determining when to end the culturing step and to harvest the myceliated low-quality protein composition, which according to the present invention, to result in a myceliated low-quality protein composition with acceptable taste, flavor and/or aroma profiles, can be determined in accordance with any one of a number of factors as defined herein, such as, for example, visual inspection of mycelia, microscope inspection of mycelia, pH changes, changes in dissolved oxygen content, amount of biomass produced, and/or assessment of taste profile, flavor profile, or aroma profile. In another embodiment, production of a certain amount of biomass may be the criteria used for harvest. For example, biomass may be measured by filtering, such through a filter of 10-1000 μm. In one embodiment, harvest can occur when the dissolved oxygen reaches about 10% to about 90% dissolved oxygen, or less than about 80% of the starting dissolved oxygen. Additionally, mycelial products may be measured as a proxy for mycelial growth, such as, total reducing sugars (usually a 40-95% reduction), R-glucan and/or ergosterol formation. Other indicators include small molecule metabolite production depending on the strain (e.g. eritadenine on the order of 0.1-20 ppm for L. edodes or erinacine on the order of 0.1-1,000 ppm for H. erinaceus) or nitrogen utilization (monitoring through the use of any nitrogenous salts or protein, may continue to culture to enhance the presence of mycelial metabolites).

Harvest includes obtaining the myceliated low-quality protein composition which is the result of the myceliation step. After harvest, cultures can be processed according to a variety of methods. In one embodiment, the myceliated low-quality protein composition is pasteurized or sterilized. In one embodiment, the myceliated low-quality protein composition is then dried according to methods as known in the art. Additionally, concentrates and isolates of the material may be prepared using variety of solvents or other processing techniques known in the art. In one embodiment the material is pasteurized or sterilized, dried and powdered by methods known in the art. Drying can be done in a desiccator, vacuum dryer, conical dryer, spray dryer, fluid bed, infrared dryer, or any method known in the art. Preferably, methods are chosen that yield a dried myceliated low-quality protein composition (e.g., a powder) with the greatest digestibility and bioavailability. The dried one or more myceliated low-quality protein composition can be optionally blended, pestled, milled or pulverized, or other methods as known in the art.

In many cases, the flavor, taste and/or aroma of one or more low-quality protein composition as disclosed herein, such as protein concentrates or isolates from vegetarian or nonvegetarian sources may have flavors, which are often perceived as unpleasant, having pungent aromas and bitter or astringent tastes. These undesirable flavors and tastes are associated with their source(s) and/or their processing, and these flavors or tastes can be difficult or impossible to mask or disguise with other flavoring agents. The present invention, as explained in more detail below, works to modulate these tastes and/or flavors.

In one embodiment of the invention, flavors and/or tastes of the myceliated low-quality protein composition are modulated as compared to the one or more ingredients (starting material). In some embodiments, both the sterilization and myceliation contribute to the modulation of the resultant myceliated low-quality protein composition's taste.

In an embodiment, the myceliated low-quality protein composition has reduced bitterness and/or reduced mustard, sulfur aroma compared to the low-quality protein composition that is not myceliated. In an embodiment, the myceliated low-quality protein composition has reduced beany, dirty/malty, cereal, bitter and/or earthy, or hay notes (dirt, woody) flavor compared to one or more low-quality protein composition that is not myceliated. For all materials, deflavoring and/or deodorizing occurs as a result of the processes of the present invention.

In an embodiment, the myceliated one or more myceliated low-quality protein composition has the changed organoleptic perception as disclosed in the present invention, as determined by human sensory testing. It is to be understood that the methods of the invention only optionally include a step of determining whether the flavor of the myceliated low-quality protein composition differs from a control material. The key determinant is, if measured by methods as disclosed herein, that the myceliated low-quality protein composition is capable of providing the named differences from control materials which have not been cultured with a fungus as named herein (e.g., sham fermentation).

Sensory evaluation is a scientific discipline that analyses and measures human responses to the composition of food and drink, e.g. appearance, touch, odor, texture, temperature and taste. Measurements using people as the instruments are sometimes necessary. The food industry had the first need to develop this measurement tool as the sensory characteristics of flavor and texture were obvious attributes that cannot be measured easily by instruments. Selection of an appropriate method to determine the organoleptic qualities, e.g., flavor, of the instant invention can be determined by one of skill in the art, and includes, e.g., discrimination tests or difference tests, designed to measure the likelihood that two products are perceptibly different. Responses from the evaluators are tallied for correctness, and statistically analyzed to see if there are more correct than would be expected due to chance alone.

In the instant invention, it should be understood that there are any number of ways one of skill in the art could measure the sensory differences.

In an embodiment, the myceliated low-quality protein composition e.g., produced by methods of the invention, has reduced bitterness, as measured by sensory testing as known in the art. Such methods include change in taste threshold, change in bitterness intensity, and the like. At least 10% or more change (e.g., reduction in) bitterness is preferred. The increase in desirable flavors and/or tastes may be rated as an increase of 1 or more out of a scale of 5 (1 being no taste, 5 being a very strong taste.) Or, a reference may be defined as 5 on a 9 point scale, with reduced bitterness or at least one flavor as 1-4 and increased bitterness or at least one flavor as 6-9.

The invention also includes wherein myceliated low-quality protein composition has less perceived flavor of raw or unfermented low-quality protein composition measured by organoleptic qualities as discussed herein. For example, corn gluten meal has attributes of being mustard yellow in color, bitter in taste, with an undesirable and unpleasant “sulfur” aroma. The aroma included sulfur, mustard and other undesirable/unpleasant volatiles. The material also has sensory attributes of being slightly sweet in taste with a dry corn and starchy flavor. The invention includes reduction in one or more of the named organoleptic qualities. For example, the treated corn gluten meal has a mild aroma, with no or reduced sulfur, mustard, or volatile notes. The treated material had a sweet, malty taste, and the color has been changed to a light brown or tan. For other low-quality protein compositions, reductions in undesirable tastes such as bitter tastes, beany tastes, earthy tastes are found and reductions in undesirable aromas such as beany, earthy, musty, or sulfur are found.

Additionally, the organoleptic qualities of the myceliated low-quality protein composition may also be improved by processes of the current invention. For example, deflavoring can be achieved, resulting in a milder flavor and/or with the reduction of, for example, bitter and/or astringent tastes. The decrease in undesirable flavors and/or tastes as disclosed herein may be rated as a decrease of 1 or more out of a scale of 5 (1 being no taste, 5 being a very strong taste.)

Culturing times and/or conditions can be adjusted to achieve the desired aroma, flavor and/or taste outcomes. As compared to the control and/or ingredient composition, and/or the pasteurized, dried and powdered medium not subjected to sterilization or myceliation, the resulting myceliated low-quality protein composition in some embodiments is less bitter and has a more mild, less sulfur/mustard aroma, and the resulting myceliated rice bran protein material is less bitter and has a more mild, less beany, malty, cereal, or earthy aroma, or less rancid, sour, or feed food notes.

Embodiments of the present invention also include a myceliated low-quality protein composition made by the methods of the invention. Embodiments also include a composition which includes a comprising a myceliated low-quality protein composition, wherein the myceliated low-quality protein composition is at least 20% (w/w) protein on a dry weight basis, wherein myceliated low-quality protein composition is myceliated by filamentous fungal culture comprising Lentinula spp., Pleurotus spp., or Morchella spp. in a media comprising at least 20 g/L protein, and wherein the myceliated low-quality protein composition has improved aroma and/or improved taste and/or decreased bitter taste, compared to a low-quality protein composition that is not myceliated.

Such prepared myceliated low-quality protein compositions can be used to create a number of food compositions, including, without limitation, spreads, pastes such as sweet (e.g. chocolate or fruit) pastes or savory pastes, prewhipped toppings, custards, coatings, peanut butter, frostings, cream filings, confectionery fillings, dairy alternative products, beverages and beverage bases, extruded and extruded/puffed products, meat imitations and extenders, baked goods and baking mixes, granola products, bar products, smoothies and juices, and soups and soup bases, all of which contain a myceliated low-quality protein compositions according to the invention. The invention includes methods to make food compositions, comprising providing a myceliated low-quality protein compositions of the invention, providing an edible material, and mixing the myceliated low-quality protein compositions of the invention and the edible material. The edible material can be, without limitation, a starch, a flour, a grain, a lipid, a colorant, a flavorant, an emulsifier, a sweetener, a vitamin, a mineral, a spice, a fiber, a protein powder, nutraceuticals, sterols, isoflavones, lignans, glucosamine, an herbal extract, xanthan, a gum, a hydrocolloid, a starch, a preservative, a legume product, a food particulate, and combinations thereof. A food particulate can include cereal grains, cereal flakes, crisped rice, puffed rice, oats, crisped oats, granola, wheat cereals, protein nuggets, texturized plant protein ingredients, flavored nuggets, cookie pieces, cracker pieces, pretzel pieces, crisps, soy grits, nuts, fruit pieces, corn cereals, seeds, popcorn, yogurt pieces, and combinations of any thereof.

The methods to prepare a food composition can include the additional, optional steps of cooking, extruding, and/or puffing the food composition according to methods known in the art to form the food compositions comprising the myceliated low-quality protein compositions of the invention.

In one embodiment, the food composition can include an alternative dairy product comprising a myceliated low-quality protein compositions according to the invention. An alternative dairy product according to the invention includes, without limitation, products such as imitation skimmed milk, imitation whole milk, imitation cream, imitation cream filling, imitation fermented milk product, imitation cheese, imitation yogurt, imitation butter, imitation dairy spread, imitation butter milk, imitation acidified milk drink, imitation sour cream, imitation ice cream, imitation flavored milk drink, or an imitation dessert product based on milk components such as custard. Methods for producing alternative dairy products using alternative proteins, such as plant-based proteins as disclosed herein including nuts (almond, cashew), seeds (hemp), legumes (pea), rice, and soy are known in the art. These known methods for producing alternative dairy products using a plant-based protein can be adapted to use with a myceliated low-quality protein compositions using art-known techniques.

The present invention can also include extruded and/or puffed products and/or cooked products comprising a myceliated low-quality protein compositions of the invention. Extruded and/or puffed ready-to-eat breakfast cereals and snacks are known in the art. Extrusion processes are well known in the art and appropriate techniques can be determined by one of skill. These materials are formulated primarily with cereal grains and may contain flours from one or more cereal grains. The composition of the present invention contain flour from at least one cereal grain, preferably selected from corn and/or rice, or alternatively, wheat, rye, oats, barley, and mixtures thereof. The cereal grains used in the present invention are commercially available, and may be whole grain cereals, but more preferably are processed from crops according to conventional processes for forming refined cereal grains. The term “refined cereal grain” as used herein also includes derivatives of cereal grains such as starches, modified starches, flours, other derivatives of cereal grains commonly used in the art to form cereals, and any combination of such materials with other cereal grains.

The food product produced using the methods described herein can be in the form of crunchy curls, puffs, chips, crisps, crackers, wafers, flat breads, biscuits, crisp breads, protein inclusions, cones, cookies, flaked products, fortune cookies, etc. The food product can also be in the form of pasta, such as dry pasta or a ready-to-eat pasta. The product can be used as or in a snack food, cereal, or can be used as an ingredient in other foods such as a nutritional bar, breakfast bar, breakfast cereal, or candy. In a pasta, the one myceliated low-quality protein compositions may be, in a non-limiting example, be used in levels of about 10 g per 58 g serving (17%).

A food composition of the invention can also include a texturized protein, such as a texturized plant protein. Texturized plant protein comprising the myceliated low-quality protein compositions of the present invention include meat imitation products and methods for making meat imitation products comprising the myceliated low-quality protein compositions as disclosed within. The myceliated low-quality protein compositions analog meat products can be produced with high moisture content and provide a product that simulates the fibrous structure of animal meat and has a desirable meat-like moisture, texture, mouthfeel, flavor and color. Methods for making such products using plant-based proteins such as pea protein, soy protein and the like are known in the art and such methods may be used in the instant invention. Texturization of protein is the development of a texture or a structure via a process involving heat, and/or shear and the addition of water. The texture or structure will be formed by protein fibers that will provide a meat-like appearance and perception when consumed. To make non-animal proteins palatable, texturization into fibrous meat analogs, for example, through extrusion processing has been an accepted approach. Due to its versatility, high productivity, energy efficiency and low cost, extrusion processing is widely used in the modern food industry. Extrusion processing is a multi-step and multifunctional operation, which leads to mixing, hydration, shear, homogenization, compression, deaeration. pasteurization or sterilization, stream alignment, shaping, expansion and/or fiber formation.

Food compositions comprising the compositions of the invention include, for example, bakery products and baking mixes comprising myceliated low-quality protein compositions according to known methods. The term “bakery product” includes, but is not limited to leavened or unleavened, traditionally flour-based products such as white pan and whole wheat breads (including sponge and dough bread), cakes, pretzels, muffins, donuts, brownies, cookies, pancakes, biscuits, rolls, crackers, pie crusts, pizza crusts, hamburger buns, pita bread, and tortillas.

Food compositions comprising the compositions of the invention also include, for example, spreads, pastes such as sweet (e.g. chocolate or fruit) pastes or savory pastes, prewhipped toppings, custards, coatings, peanut butter, frostings, cream filings, confectionery fillings and other confectioneries.

The present invention also includes food compositions such as granola cereals, and bar products, including such as granola bars, nutrition bars, energy bars, sheet and cut bars, extruded bars, baked bars, and combinations thereof.

The baked food compositions and bar compositions are generally formed dependent on the desired end product. The baked food compositions and bar compositions are produced according to standard industry recipes, substituting in a myceliated one or more low-quality protein composition food product of the present invention for at least some of the called-for sugar and/or fat ingredients.

In one embodiment, the invention includes preparation of spreads that have increased nutritional content, for example a relatively high protein content. The nutritional paste includes combining one or more myceliated low-quality proteins of the present invention, together with fats and emulsifiers to form said paste; wherein the paste has a low water activity and low pH to substantially prevent bacterial growth and enable the paste to be stable without being stored at 4° C. Preferably the paste has 10 to 30% w/w myceliated low-quality proteins. The myceliated low-quality protein of the present invention can be mixed with, optionally, for a chocolate-flavored paste, sweet ingredients, chocolate-flavor ingredients and fat/emulsifier ingredients to form a premix and milling or blending to a smooth paste, as known in the art. Optionally, the fat ingredient may include a nut butter, but may also include any vegetable fat such as, for example, cocoa butter, palm, palm kernel, soybean, safflower, cottonseed, coconut, rapeseed, canola, corn, peanut and sunflower oils, or mixtures thereof. High melting vegetable oil stabilizers of palm, cottonseed and similar vegetable oil origins at a level of 0.5-10% may be used.

The following examples further illustrate the invention but, of course, should not be construed as in any way limiting its scope.

### EXAMPLES

### Example 1

Eighteen (18) 1 L baffled DeLong Erlenmeyer flasks were filled with 0.400 L of a medium consisting of 25 g/L organic pea protein concentrate (labeled as 80% protein), 25 g/L organic rice protein concentrate (labeled as 80% protein), 4 g/L organic dry malt extract, 2 g/L diammonium phosphate, 1 g/L organic carrot powder and 0.4 g/L magnesium sulfate heptahydrate in RO water. The flasks were covered with a stainless steel cap and sterilized in an autoclave on a liquid cycle that held the flasks at 120-121° C. for 1 hour. The flasks were carefully transferred to a clean HEPA laminar flowhood where they cooled for 18 hours. Sixteen (16) flasks were subsequently inoculated with 2 cm² pieces of mature Petri plate cultures of P. ostreatus, P. eryngii, L. nuda, H. erinaceus, L. edodes, A. blazeii, L. sulfureus and B. edulis, each strain done in duplicate from the same plate. All 18 flasks were placed on a shaker table at 150 rpm with a swing radius of 1″ at room temperature. The Oyster (P. ostreatus), Blewit (Lepista nuda) and Lion's Mane (H. erinaceus) cultures were all deemed complete at 72 hours by way of visible and microscopic inspection (mycelial balls were clearly visible in the culture, and the isolation of these balls revealed dense hyphal networks under a light microscope). The other samples, but for the Porcini (Boletus edulis) which did not grow well, were harvested at 7 days. All samples showed reduced pea and reduced rice aroma and flavor, as well as less “beany” type aromas/flavors. The Oysters had a specifically intense savory taste and back-end mushroom flavor. The Blewit was similar but not quite as savory. The Lion's Mane sample had a distinct ‘popcorn’ aroma. The 3, 7 day old samples were nearly considered tasteless but for the Chicken of the Woods (Laetiporus sulphureus) sample product which had a nice meaty aroma and had no pea or rice aroma/flavor. The control sample smelled and tasted like a combination of pea and rice protein and was not considered desirable. The final protein content of every the resulting cultures was between 50-60% and the yields were between 80-90% after desiccation and pestling.

### Example 2

Three (3) 4 L Erlenmeyer flasks were filled with 1.5 L of a medium consisting of 5 g/L pea protein concentrate (labeled as 80% protein), 5 g/L rice protein concentrate (labeled as 80% protein), 3 g/L malt extract and 1 g/L carrot powder. The flasks were wrapped with a sterilizable biowrap which was wrapped with autoclave tape 5-6 times (the taped biowrap should be easily taken off and put back on the flask without losing shape) and sterilized in an autoclave that held the flasks at 120-121° C. for 1 hour. The flasks were carefully transferred to a clean HEPA laminar flowhood where they cooled for 18 hours. Each flask was subsequently inoculated with 2 cm2 pieces of 60 day old P1 Petri plate cultures of L. edodes and placed on a shaker table at 120 rpm with a 1″ swing radius at 26° C. After 7-15 days, the inventors noticed, by using a pH probe on 20 mL culture aliquots, that the pH of every culture had dropped nearly 2 points since inoculation. L. edodes is known to produce various organic acids on or close to the order of g/L and the expression of these acids are likely what dropped the pH in these cultures. A microscope check was done to ensure the presence of mycelium and the culture was plated on LB media to ascertain the extent of any bacterial contamination. While this culture could have been used as a food product with further processing (pasteurization and optionally drying), the inventors typically use such cultures as inoculant for bioreactor cultures of media prepared as disclosed according to the methods of the present invention.

### Example 3

A 7 L bioreactor was filled with 4.5 L of a medium consisting of 5 g/L pea protein concentrate (labeled as 80% protein), 5 g/L rice protein concentrate (labeled as 80% protein), 3 g/L malt extract and 1 g/L carrot powder. Any open port on the bioreactor was wrapped with tinfoil and sterilized in an autoclave that held the bioreactor at 120-121° C. for 2 hours. The bioreactor was carefully transferred to a clean bench in a cleanroom, setup and cooled for 18 hours. The bioreactor was inoculated with 280 mL of inoculant from a 12 day old flask as prepared in Example 2. The bioreactor had an air supply of 3.37 L/min (0.75 VVM) and held at 26° C. A kick-in/kick-out antifoam system was setup and it was estimated that ˜1.5 g/L antifoam was added during the process. At ˜3-4 days the inventors noticed that the pH of the culture had dropped ˜1.5 points since inoculation, similar to what was observed in the flask culture. A microscope check was done to ensure the presence of mycelium (mycelial pellets were visible by the naked eye) and the culture was plated on LB media to ascertain the extent of any bacterial contamination and none was observed. While this culture could have been used as a food product with further processing (pasteurization and optionally drying), the inventors typically use such cultures as inoculant for bioreactor cultures of media prepared as disclosed according to the methods of the present invention.

### Example 4

A 250 L bioreactor was filled with 150 L of a medium consisting of 45 g/L pea protein concentrate (labeled as 80% protein), 45 g/L rice protein concentrate (labeled as 80% protein), 1 g/L carrot powder, 1.8 g/L diammonium phosphate, 0.7 g/L magnesium sulfate heptahydrate, 1 g/L antifoam and 1.5 g/L citric acid and sterilized in place by methods known in the art, being held at 120-121° C. for 100 minutes. The bioreactor was inoculated with 5 L of inoculant from two bioreactors as prepared in Example 3. The bioreactor had an air supply of 30 L/min (0.2 VVM) and held at 26° C. The culture was harvested in 4 days upon successful visible (mycelial pellets) and microscope checks. The pH of the culture did not change during processing but the DO dropped by 25%. The culture was plated on LB media to ascertain the extent of any bacterial contamination and none was observed. The culture was then pasteurized at 82° C. for 30 minutes with a ramp up time of 30 minutes and a cool down time of 45 minutes to 17° C. The culture was finally spray dried and tasted. The final product was noted to have a mild aroma with no perceptible taste at concentrations up to 10%. The product was ˜75% protein on a dry weight basis.

### Example 5

A 250 L bioreactor was filled with 200 L of a medium consisting of 45 g/L pea protein concentrate (labeled as 80% protein), 45 g/L rice protein concentrate (labeled as 80% protein), 1 g/L carrot powder, 1.8 g/L diammonium phosphate, 0.7 g/L magnesium sulfate heptahydrate, 1 g/L antifoam and 1.5 g/L citric acid and sterilized in place by methods known in the art, being held at 120-121° C. for 100 minutes. The bioreactor was inoculated with 5 L of inoculant from two bioreactors as prepared in Example 3. The bioreactor had an air supply of 30 L/min (0.2 VVM) and held at 26° C. The culture was harvested in 2 days upon successful visible (mycelial pellets) and microscope checks. The pH of the culture did not change during processing but the DO dropped by 25%. The culture was plated on LB media to ascertain the extent of any bacterial contamination and none was observed. The culture was then pasteurized at 82° C. for 30 minutes with a ramp up time of 30 minutes and a cool down time of 90 minutes to 10° C. The culture was finally concentrated to 20% solids, spray dried and tasted. The final product was noted to have a mild aroma with no perceptible taste at concentrations up to 10%. The product was ˜75% protein on a dry weight basis.

### Example 6

Eight (8) 1 L baffled DeLong Erlenmeyer flasks were filled with 0.4 L of media consisting of 45 g/L pea protein concentrate (labeled as 80% protein), 45 g/L rice protein concentrate (labeled as 80% protein), 1 g/L carrot powder, 1 g/L malt extract, 1.8 g/L diammonium phosphate and 0.7 g/L magnesium sulfate heptahydrate and sterilized in an autoclave being held at 120-121° C. for 1 hour. The flasks were then carefully placed into a laminar flowhood and cooled for 18 hours. Each flask was inoculated with 240 mL of culture as prepared Example 2 except the strains used were G. lucidum, C. sinensis, I. obliquus and H. erinaceus, with two flasks per species. The flasks were shaken at 26° C. at 120 RPM with a 1″ swing radius for 8 days, at which point they were pasteurized as according to the parameters discussed in Example 5, desiccated, pestled and tasted. The G. lucidum product contained a typical ‘reishi’ aroma, which most of the tasters found pleasant. The other samples were deemed pleasant as well but had more typical mushroom aromas.

As compared to the control, the pasteurized, dried and powdered medium not subjected to sterilization or myceliation, the resulting myceliated food products was thought to be much less bitter and to have had a more mild, less beany aroma that was more cereal in character than beany by 5 tasters. The sterilized but not myceliated product was thought to have less bitterness than the nonsterilized control but still had a strong beany aroma. The preference was for the myceliated food product.

### Example 7

A 4,000 L bioreactor was filled with 2,500 L of a sterilized medium similar to Example 4, consisting of 45 g/L pea protein concentrate (labeled as 80% protein), 45 g/L rice protein concentrate (labeled as 80% protein), 3.6 g/l maltodextrin, 1.8 g/L carrot powder, 1.8 g/L diammonium phosphate, 0.7 g/L magnesium sulfate heptahydrate, 1.5 g/L antifoam and 0.6 g/L citric acid. Seed reactor was also prepared in 200 L bioreactor with medium volume of 100 L with the following medium components: pea protein 5 g/l, rice protein 5 g/l, maltodextrin 3.0 g/l, carrot powder 1 g/l, malt extract 3 g/l and 1.25 g/l of antifoam. The medium was inoculated with flask process developed the same way as shown in Example 2. Inoculum was harvested when pH was 4.7+/−0.1. The 200 L bioreactor was harvested 55 hours post-inoculation. The flasks were harvested 11 days post-inoculation. The organism was Lentinula edodes sourced from the Penn State mushroom culture collection.

Once the main fermenter was cooled it was inoculated with the 100 L inoculum from the 200 L fermentor. The culture in the 4,000 L vessel was harvested at 48 hours post-inoculation upon successful visible (mycelial pellets) and microscope checks. No pH change was observed during the fermentation. Material was pasteurized in the bioreactor at 65 C for 60 minutes. Fermenter was then cooled down and material was harvested in sanitized 55 gallon drums and sent to spray drying facility.

### Example 8

A 10,000-L bioreactor was prepared with the following medium components for a working volume of 6,200 L. pea protein 45 g/l, rice protein 45 g/l, maltodextrin 3.6 g/l, carrot powder 1.8 g/l, magnesium sulfate 0.72 g/l, di ammonium phosphate 1.8 g/l, citric acid 0.6 g/l, and 1.25 g/l of antifoam added at the end of the charge. Medium was sterilized for 2 hours at 126° C. Agitation was maintained to get a tip speed of 0.88 m/sec. Additional antifoam of 0.25 g/l was added to contain the foaming. pH of the medium remained at 6.1 throughout the fermentation. Temperature for the fermentation as maintained at 26° C. Pressure in the fermenter was increased from 0.1 bar to 1.2 bar during the course of fermentation to minimize the foaming. Fermentation was completed in 45-50 hours. After completion of fermentation the fermented broth was pasteurized and concentrated to 20% and then spray dried.

The seed inoculum for the fermentation was prepared in a 2000 L fermentor with a working volume of 530-540 L with the following medium: pea protein 5 g/l, rice protein 5 g/l, maltodextrin 3.0 g/l, carrot powder 1 g/l, malt extract 3 g/l and 1.5 g/l of antifoam. Fermentation pH was at 5.7 at the beginning of the fermentation. Fermentation was performed for 60 to 70 hours when pH reached between 4.6 and 4.9. The tip speed in the fermenter was maintained at 0.5-0.6 m/s. Aeration was done at 0.65-0.75 vvm. Fermenter was maintained at a pressure of 0.4-0.6 bar. Seed 1 for the inoculation of fermenter 2 was prepared in 150 L with a working volume of 55-65 L with the following medium: pea protein 5 g/l, rice protein 5 g/l, maltodextrin 3.0 g/l, carrot powder 1 g/l, malt extract 3 g/l, mango puree 3 g/l and 1.5 g/l of antifoam. Fermentation pH was at 5.7 at the beginning of the fermentation. The tip speed in the fermenter was maintained at 0.69 m/s and pressure was maintained at 0.5 bar. Aeration was done at a rate of 0.75 vvm. The initial pH for the fermentation was at 5.7. Fermentation was completed between 45 and 55 hours. Inoculum for Seed 1 was prepared with the 5 flask prepared in 3 L flask with the following medium: Pea Protein 5 g/l, Rice Protein 5 g/l, Maltodextrin 3.0 g/l, Carrot Powder 1 g/l, malt extract 3 g/l, mango puree 3 g/l and 1.25 g/l of antifoam. Flask were inoculated with 4 cm² agar and incubated between 11 and 13 days. pH of the flask was obtained at 4+/−2.

### Example 9

The medium for 180,000 L bioreactor was prepared as a volume of 120,000 L with the following components: pea protein 45 g/l (labeled as 80% protein), rice protein 45 g/l (labeled as 80% protein), maltodextrin 3.6 g/l, carrot powder 1.8 g/l, magnesium sulfate 0.72 g/l, di ammonium phosphate 1.8 g/l, citric acid 0.6 g/l, and 1.25 g/l of antifoam added at the end of the charge. The 180,000 L bioreactor was harvested at 48 hours.

The inoculum for the 180,000 L bioreactor was 6,200 L from a 10,000 L bioreactor prepared similar to the medium of Example 3. The 6,200 L bioreactor in turn was inoculated with 65 L of culture in a 150 L bioreactor prepared similar to the 6,200 L medium and was cultured to just before stationary phase. The 65 L medium was inoculated with flasks of Lentinula edodes in medium similar to that of the medium of Example 3 and cultured to stationary phase. These flasks had been inoculated with Lentinula edodes from the Penn State mushroom culture collection and culture to stationary phase.

### Example 10

Eight protein powders were tested: (a) raw material (3.2 pea); (b) raw material (pea); (c) raw material (rice); (d) raw material (rice); (e) myceliated material 3; (f) myceliated material 4; (g) myceliated material 4.2; and (h) myceliated material 3.2. Each protein powder was tested at 7% in water. Trained descriptive panelists used a consensus descriptive analysis technique to develop the language, ballot and rate profiles of the protein powders. The aroma language was as follows:

Overall aroma: the intensity of the total combined aroma; pea aroma, the aroma of dried peas/pea starch (reference; ground dried peas); beany aroma, the aroma of beans/bean starch (reference; ground dried lentils); rice aroma, the aroma of white rice (reference, cooked minute rice); mushroom aroma, the aroma of mushrooms (reference, dried shiitake mushrooms); overripe vegetable aroma, the aroma of soft overripe vegetables; and cardboard aroma, the aroma of pressed wet cardboard (reference: wet pressed cardboard).

The taste language was as follows: sweet, taste on the tongue stimulated by sugar in solution (reference, Domino Sugar in distilled water); sour, acidic taste on the tongue associated with acids in solution (reference, citric acid in distilled water); umami, the savory taste of MSG (reference; MSG in distilled water); bitter, basic taste on tongue associated with caffeine solutions (reference, caffeine powder in distilled water); astringent, the drying, puckering feeling associated with tannins (reference Mott's Apple Juice (40) Welch's Grape Juice (75)).

Flavor language was as follows: overall flavor, the composite intensity of all flavors as experienced while drinking the product; overripe vegetable, the flavor of soft overripe vegetables; pea, the flavor of dried peas/pea starch (reference: ground dried peas); beany, the flavor of beans/bean starch (reference: ground dried lentils; canned garbanzo beans); rice, the flavor of white rice (reference: cooked minute rice); mushroom, the flavor of mushrooms (reference: dried shiitake mushrooms); soapy, reminiscent of soap; chalky, the flavor associated with chalk and calcium (reference: citrucel gummies); cardboard, the flavor of pressed wet cardboard (reference: wet pressed cardboard); earthy, the flavor of fresh earth/dirt (reference: potting soil).

The raw pea product prior to myceliation has a pea aroma with no rice or mushroom aroma. The rice samples prior to myceliation have rice aroma with no pea or mushroom aroma. After myceliation, these samples have mushroom aroma and no pea or rice aroma, respectively. There is also increased umami flavor in the myceliated samples.

### Example 11

Eight (8) 1 L baffled DeLong Erlenmeyer flasks were filled with 0.500 L of the following 8 different media, see Table 1, after the manner of Example 1:

TABLE 1

| Component | Medium 1 | Medium 2 | Medium 3 | Medium 4 | Medium 5 | Medium 6 | Medium 7 | Medium 8 |
|---|---|---|---|---|---|---|---|---|
| Pea protein 1 | 54 | 54 | 49.5 | 54 | 54 | 54 | 0 | 54 |
| (g/L) | | | | | | | | |
| Chickpea powder | 36 | 36 | 22.5 | 36 | 36 | 36 | 36 | 36 |
| (g/L) | | | | | | | | |
| Rice protein | 0 | 0 | 18 | 0 | 0 | 0 | 0 | 0 |
| (g/L) | | | | | | | | |
| Magnesium | 0.72 | 0.72 | 0.72 | 0.72 | 0.72 | 0.72 | 0.72 | 0.72 |
| sulfate (g/L) | | | | | | | | |
| Diammonium | 1.8 | 1.8 | 1.8 | 1.8 | 1.8 | 1.8 | 1.8 | 1.8 |
| phosphate (g/L) | | | | | | | | |
| Citric acid (g/L) | 1.5 | 1.5 | 1.5 | 1.5 | 0.6 | 0.9 | 1.5 | 1.5 |
| Carrot powder | 1.8 | 1.8 | 1.8 | 1.8 | 1.8 | 1.8 | 0 | 1.8 |
| (g/L) | | | | | | | | |
| Antifoam 1 (g/L) | 1.25 | 0 | 1.25 | 1.25 | 1.25 | 1.25 | 1.25 | 1.25 |
| Pea protein 2 | 0 | 0.1 | 0 | 0 | 0 | 0 | 54 | 0 |
| (g/L) | | | | | | | | |
| Antifoam 2 (g/L) | 0 | 0.1 | 0 | 0 | 0 | 0 | 0 | 0 |
| Vegetable juice | 0 | 0 | 0 | 0 | 0 | 0 | 5 | 0 |
| (mL/L) | | | | | | | | |

The flasks were covered with a stainless-steel cap and steam sterilized. The flasks were carefully transferred to a clean HEPA laminar flow hood where they cooled for 4 hours and each were inoculated with 5% of 10-day old submerged Lentinula edodes. All 8 flasks were placed on a shaker table at 150 rpm with a swing radius of 1″ at room temperature and allowed to incubate for 3 days. Plating aliquots of each sample on LB and petri film showed no contamination in any flask. The pH changes during processing is shown below, and is essentially the same (within the margin of error of the pH meter).

Top performing recipes in sensory from these 8 media were media 5 and 7. Bitterness and sourness were evaluated and these two media showed the best results, although all media exhibited reduced undesirable flavors and reduced aromas. The sensory evaluation included 15 tasters, all tasting double-blind, randomized samples and providing a descriptive analysis. These recipes were further evaluated for strain screening work as described in Example 2.

### Example 12

Eight (8) 1 L baffled DeLong Erlenmeyer flasks were filled with 0.500 L of medium consisting of the 2 best medium as described in example 1 (4 flasks for each medium), see Table 2. These two media were inoculated with four different species: Lentinula edodes, Boletus edulis, Pleurotus salmoneostramineus and Morchella esculenta.

TABLE 2

| Component | Medium 1 | Medium 2 |
|---|---|---|
| Pea protein 1 (g/L) | 54 | 0 |
| Chickpea powder (g/L) | 36 | 36 |
| Magnesium sulfate (g/L) | 0.72 | 0.72 |
| Diammonium phosphate (g/L) | 1.8 | 1.8 |
| Citric Acid (g/L) | 0.6 | 1.5 |
| Carrot powder (g/L) | 1.8 | 1.8 |
| Pea protein 2 (g/L) | 0 | 54 |
| Antifoam 2 (g/L) | 0.1 | 0.1 |

The flasks were covered with a stainless-steel cap and sterilized in an autoclave. The flasks were carefully transferred to a clean HEPA laminar flow hood where they cooled for 4 hours and inoculated with 5% of 10-day old submerged aliquots of each species. All 8 flasks were placed on a shaker table at 150 rpm with a swing radius of 1″ at room temperature and incubated for 3 days.

Plating aliquots of each sample on petri film showed no contamination in any flask. Bitterness and sourness were evaluated and these two media showed the best results, although all media exhibited reduced undesirable flavors and reduced aromas. The results that were obtained showed that Boletus edulis performed better than other species for lower sourness and bitterness.

### Example 13

One (1) 1 L baffled DeLong Erlenmeyer flask was filled with 0.500 L of a medium consisting of the following recipe in Table 3.

TABLE 3

| Component | Medium |
|---|---|
| Corn gluten meal (g/L) | 90 |
| Magnesium sulfate (g/L) | 0.72 |
| Diammonium phosphate (g/L) | 1.8 |
| Carrot powder (g/L) | 1.8 |
| Antifoam 2 (g/L) | 0.5 |

The flask was covered with a stainless-steel cap and sterilized in an autoclave. The flask was carefully transferred to a clean HEPA laminar flow hood where it cooled for 4 hours and was then inoculated with 5% of 13-day old submerged aliquots of Lentinula edodes. This flask was placed on a shaker table at 150 rpm with a swing radius of 1″ at room temperature and incubated for 7 days at 26° C. The pH change of up to 0.2 units was observed for this myceliation. Mycelium growth was observable. The corn gluten meal (raw) had sensory characteristics as follows: aroma, mustard, sulfur. Color: yellow. Taste: bitter, slightly sweet, dry corn, starchy. The myceliated corn gluten meal had the following characteristics: mild smell, no mustard or sulfur aroma. Color is light brown/tan. Taste: reduced bitterness, sour, fermented, malty.

### Example 14

Two 1 L baffled DeLong Erlenmeyer flasks were filled with 0.500 L of a medium consisting of 90 g/l of corn gluten meal. The flasks were covered with a stainless-steel cap and sterilized in an autoclave on a liquid cycle that held the flasks at 120-123° C. for 1.5 hour. The flasks were carefully transferred to a clean HEPA laminar flow hood where they cooled for 4 hours and were inoculated with 10% culture of Lentinula edodes grown on medium consisting of 25 g/L glucose, 5 g/L yeast extract. These flasks were placed on a shaker table at 150 rpm with a swing radius of 1″ at room temperature and incubated for 7 days. Samples were examined under microscope at the end of 7 days fermentation. A microscope check was done to ensure the presence of mycelium (mycelial pellets were visible by the naked eye) and the culture was plated on LB media to ascertain the extent of any bacterial contamination and none was observed. These cultures were dried at 65° C. and organoleptic tasting was conducted to determine how it differed from raw corn gluten meal.

The pH of the corn gluten meal was as summarized as follows: initial post inoculation pH: 4.24; Final Harvest pH on day 7: 4.15. Sensory data can be seen in Table 4.

TABLE 4

| | |
|---|---|
| Sample: Corn Gluten | Mustard yellow color, terrible aroma, corny, |
| Meal Raw | dirt like flavor sulfur like notes. |
| Sample: Corn Gluten | Light brown/yellow, mushroomy flavor, |
| Mean Fermented | much improved aroma, dirty notes, |
| | slightly metallic off notes, less |
| | corn flavor. |

These results clearly suggest significant change in color, flavor and aroma as sulfur flavor was completely eliminated.

### Example 15

One (1) 1 L baffled DeLong Erlenmeyer flask was filled with 0.500 L of a medium consisting of the following recipe in Table 5:

TABLE 5

| Component | Medium |
|---|---|
| Fava bean protein isolate (g/L) | 90 |
| Magnesium sulfate (g/L) | 0.72 |
| Diammonium phosphate (g/L) | 1.8 |
| Carrot powder (g/L) | 1.8 |
| Antifoam 2 (g/L) | 0.5 |

Fava bean protein isolate was obtained from Advanta Fava, 85%-90% protein. The flask was covered with a stainless-steel cap and sterilized in an autoclave. The flask was carefully transferred to a clean HEPA laminar flow hood where it cooled for 4 hours and was then inoculated with 10% of 13-day old submerged aliquots of Lentinula edodes (25 g/L glucose, 5 g/L yeast extract and 2 g/L lecithin emulsion in oil. This flask was placed on a shaker table at 150 rpm with a swing radius of 1″ at room temperature and incubated for 7 days at 26° C. Mycelium growth was observable. The cultures were dried and organoleptic tasting was conducted to determine how the cultured fava bean protein differed from the control fava bean raw protein. The initial post inoculation pH was 6.1 and the pH at harvest was 6.02. The fava bean protein (raw) had sensory characteristics as follows: aroma: slight beany, bitter, low aroma. Color: eggshell, off-white, cream. Taste: chalky, pea-like, legume, earthy, slight bean flavor, starch, sweet, bitter end. The myceliated fava bean protein had the following characteristics: umami, slightly sour. Color is sandy brown. Taste: neutral, umami, earthy (no bean taste).

### Example 16

One (1) 1 L baffled DeLong Erlenmeyer flask was filled with 0.500 L of a medium consisting of the following recipe in Table 6:

TABLE 6

| Component | Medium |
|---|---|
| Fava bean protein isolate (g/L) | 70 |
| Magnesium sulfate (g/L) | 0.72 |
| Diammonium phosphate (g/L) | 1.8 |
| Carrot powder (g/L) | 1.8 |
| Antifoam 2 (g/L) | 0.5 |

Fava bean protein isolate was obtained from Advanta Fava, 85%-90% protein. The flask was covered with a stainless-steel cap and sterilized in an autoclave. The flask was carefully transferred to a clean HEPA laminar flow hood where it cooled for 4 hours and was then inoculated with 10% of 13-day old submerged aliquots of Lentinula edodes (25 g/L glucose, 5 g/L yeast extract and 2 g/L lecithin emulsion in oil. This flask was placed on a shaker table at 150 rpm with a swing radius of 1″ at room temperature and incubated for 7 days at 26° C. Mycelium growth was observable. The cultures were dried and organoleptic tasting was conducted to determine how the cultured fava bean protein differed from the control fava bean raw protein. The initial post inoculation pH was 6.1 and the pH at harvest was 5.5. The fava bean protein (raw) had sensory characteristics as follows: aroma: slight beany, bitter, low aroma. Color: eggshell, off-white, cream. Taste: chalky, pea-like, legume, earthy, slight bean flavor, starch, sweet, bitter end. The myceliated fava bean protein had the following characteristics: umami, slightly sour. Color is sandy brown. Taste: neutral, umami, earthy (no bean taste).

### Example 17

Two (2) 1 L baffled DeLong Erlenmeyer flask was filled with 0.500 L of a medium consisting of the following recipe in Table 7:

TABLE 7

| Component | Medium |
|---|---|
| Rice bran protein (25% protein dry weight) (g/L) | 70 |
| Magnesium sulfate (g/L) | 0.72 |
| Diammonium phosphate (g/L) | 1.8 |
| Carrot powder (g/L) | 1.8 |
| Antifoam 2 (g/L) | 0.5 |

Rice bran protein was obtained from Rice Bran Tech, approximately 25% protein. The flask was covered with a stainless-steel cap and sterilized in an autoclave. The flask was carefully transferred to a clean HEPA laminar flow hood where it cooled for 4 hours and was then inoculated with 10% of 13-day old submerged aliquots of Lentinula edodes (25 g/L glucose, 5 g/L yeast extract and 2 g/L lecithin emulsion in oil. This flask was placed on a shaker table at 150 rpm with a swing radius of 1″ at room temperature and incubated for 7 days or 4 days at 26° C. Mycelium growth was observable. The cultures were dried and organoleptic tasting was conducted to determine how the cultured rice bran protein differed from the control fava bean raw protein. The initial post inoculation pH was 4.77 and the pH at harvest was 4.32. The rice bran protein (raw) had sensory characteristics as follows: aroma: beany, malty, cereal, earthy/dirt/woody. Also detected were sour, feed food, and rancid. Taste: beany, malty, cereal, bitter, earthy/dirt/woody. The myceliated fava bean protein had the following characteristics: aroma: neutral, fermented. Taste: neutral, umami, earthy (no bean taste).

### Example 18

Two 1 L baffled DeLong Erlenmeyer flasks were filled with 0.500 L of a medium consisting of 90 g/l of spent beer grains. The flasks were covered with a stainless-steel cap and sterilized in an autoclave on a liquid cycle that held the flasks at 120-123° C. for 1.5 hour. The flasks were carefully transferred to a clean HEPA laminar flow hood where they cooled for 4 hours and were inoculated with 10% aqueous culture of Lentinula edodes grown on a medium consisting of 25 g/L glucose, 5 g/L yeast extract and 1 g/l of lecithin emulsion in oil. These flasks were placed on a shaker table at 150 rpm with a swing radius of 1 inch at room temperature and incubated for 7 days. Samples were examined under microscope at the end of 7 days fermentation. A microscope check was done to ensure the presence of mycelium (mycelial pellets were visible by the naked eye) and the culture was plated on LB media to ascertain the extent of any bacterial contamination and none was observed. These cultures were dried, ground and organoleptic tasting was conducted to determine how it differed from raw control.

Sample: Beer Grains Fermented. Color—dirt brown Aroma—sweet, browned bread Flavor—sweet, caramelized, bready, yeasty, slight bitter, malty

The sensory group consensus suggests that the fermented beer grains had sweet brown/caramelized notes, with some malty/yeasty notes. It was a preferred sample due to low bitterness and its mostly sweet and bready profile.

### Example 19

GrapeSeed—90 g/L 7 day

Two 1 L baffled DeLong Erlenmeyer flasks were filled with 0.500 L of a medium consisting of 90 g/l of grapeseed powder (grapeseed powder from meal after oil processing). The flasks were covered with a stainless-steel cap and sterilized in an autoclave on a liquid cycle that held the flasks at 120-123° C. for 1.5 hour. The flasks were carefully transferred to a clean HEPA laminar flow hood where they cooled for 4 hours and were inoculated with 10% culture of Lentinula edodes grown on an aqueous medium consisting of 25 g/L glucose, 5 g/L yeast extract and 1 g/l of lecithin emulsion in oil. These flasks were placed on a shaker table at 150 rpm with a swing radius of 1″ at room temperature and incubated for 7 days. Samples were examined under microscope at the end of 7 days fermentation. A microscope check was done to ensure the presence of mycelium (mycelial pellets were visible by the naked eye) and the culture was plated on LB media to ascertain the extent of any bacterial contamination and none was observed. These cultures were dried, and organoleptic tasting was conducted to determine how it differed from raw control.

Sample: Raw Color—Cocoa powder, chocolate Aroma—sweet, earthy, cereal, resin, root vegetables Flavor—Sweet, cardboard, grape, dirty, earthy, astringent earthy, cereal, sweet, grape backend, bitter, fruity

Sample: Fermented Color—slightly darker brown Aroma—Very slightly earthy, dirty, play-doh Flavor—sweet up front, astringent, grape skin, fruit forward, most flavor/complexity, eliminates dirt notes.

Grapeseed was a preferred sample. The driver for preference may be due to the maintained sweetness, slight grape flavor, and flavor complexity. Fermentation introduced bitterness and sourness, but removed dirty/earthy notes.

GrapeSeed—90 g/L 4 day

Two 1 L baffled DeLong Erlenmeyer flasks were filled with 0.500 L of a medium consisting of 90 g/l of grapeseed powder. The flasks were covered with a stainless-steel cap and sterilized in an autoclave on a liquid cycle that held the flasks at 120-123° C. for 1.5 hour. The flasks were carefully transferred to a clean HEPA laminar flow hood where they cooled for 4 hours and were inoculated with 10% culture of Lentinula edodes grown on medium consisting of 25 g/L glucose, 5 g/L yeast extract and 1 g/l of lecithin emulsion in oil. These flasks were placed on a shaker table at 150 rpm with a swing radius of 1″ at room temperature and incubated for 7 days. Samples were examined under microscope at the end of 4 days fermentation. A microscope check was done to ensure the presence of mycelium (mycelial pellets were visible by the naked eye) and the culture was plated on LB media to ascertain the extent of any bacterial contamination and none was observed. These cultures were dried, and organoleptic tasting was conducted to determine how it differed from raw control.

Sample: Raw Color—Cocoa powder, chocolate Aroma—Earthy, sweet, grape, Play-doh Texture—Sandy, gritty, crunchy Flavor—Sweet, cardboard, grape, dirty, earthy, astringent

Sample: Fermented Color—Cocoa powder, chocolate Aroma—Stale, dirty Texture-Sandy, gritty, clusters more than raw Flavor-, astringent, grape, sweet

Grapeseed was a preferred sample. The driver for preference may be due to the maintained sweetness and slight grape flavor. Fermentation removed dirty/earthy notes.

### Example 20

Rice Bran Fiber

Two 1 L baffled DeLong Erlenmeyer flasks were filled with 0.500 L of a medium consisting of 90 g/l of rice bran fiber. The flasks were covered with a stainless-steel cap and sterilized in an autoclave on a liquid cycle that held the flasks at 120-123° C. for 1.5 hour. The flasks were carefully transferred to a clean HEPA laminar flow hood where they cooled for 4 hours and were inoculated with 10% culture of Lentinula edodes grown on medium consisting of 25 g/L glucose, 5 g/L yeast extract and 1 g/l of lecithin emulsion in oil. These flasks were placed on a shaker table at 150 rpm with a swing radius of 1″ at room temperature and incubated for 7 days. Samples were examined under microscope at the end of 7 days fermentation. A microscope check was done to ensure the presence of mycelium (mycelial pellets were visible by the naked eye) and the culture was plated on LB media to ascertain the extent of any bacterial contamination and none was observed. These cultures were dried and organoleptic tasting was conducted to determine how it differed from raw control.

Sample: Raw Color—Khaki/Tan Aroma—Cereal, sweet bean Flavor—Starchy, malty, toasted cereal, cardboard, low upfront flavor.

Sample: Fermented Color—tan sand Aroma—Slight caramelized, roasted smell, graham crackers, saw dust Flavor—Graham cracker, toasted spices, sweet grain

Overall, cereal notes were reduced after fermentation. A graham cracker, sweet grain flavor was developed.

### Example 21

Chicory—90 g/L 7 day

Two 1 L baffled DeLong Erlenmeyer flasks were filled with 0.500 L of a medium consisting of 90 g/l of chicory root powder. The flasks were covered with a stainless-steel cap and sterilized in an autoclave on a liquid cycle that held the flasks at 120-123° C. for 1.5 hour. The flasks were carefully transferred to a clean HEPA laminar flow hood where they cooled for 4 hours and were inoculated with 10% culture of Lentinula edodes grown on medium consisting of 25 g/L glucose, 5 g/L yeast extract and 1 g/l of lecithin emulsion in oil. These flasks were placed on a shaker table at 150 rpm with a swing radius of 1″ at room temperature and incubated for 7 days. Samples were examined under microscope at the end of 7 days fermentation. A microscope check was done to ensure the presence of mycelium (mycelial pellets were visible by the naked eye) and the culture was plated on LB media to ascertain the extent of any bacterial contamination and none was observed. These cultures were dried and organoleptic tasting was conducted to determine how it differed from raw control.

Sample: Raw Color—Off-white Aroma—cereal Flavor—sweet cereal, bitter on backend

Sample: Fermented Color—light brown Aroma—sharply, malty, sweet, caramel Flavor—sweet, sour, fermented, malty, caramelized, barnyard.

Overall, sweet cereal notes were converted to malty, sweet caramelized, barnyard, fermented notes.

Chicory—90 g/L 4 day

Two 1 L baffled DeLong Erlenmeyer flasks were filled with 0.500 L of a medium consisting of 90 g/l of chicory root powder. The flasks were covered with a stainless-steel cap and sterilized in an autoclave on a liquid cycle that held the flasks at 120-123° C. for 1.5 hour. The flasks were carefully transferred to a clean HEPA laminar flow hood where they cooled for 4 hours and were inoculated with 10% culture of Lentinula edodes grown on medium consisting of 25 g/L glucose, 5 g/L yeast extract and 1 g/l of lecithin emulsion in oil. These flasks were placed on a shaker table at 150 rpm with a swing radius of 1″ at room temperature and incubated for 7 days. Samples were examined under microscope at the end of 4 days fermentation. A microscope check was done to ensure the presence of mycelium and the culture was plated on LB media to ascertain the extent of any bacterial contamination and none was observed. These cultures were dried and organoleptic tasting was conducted to determine how it differed from raw control.

Sample: Raw Color—Beige Aroma—Play-doh, oat, grain, chalky Texture—Chalky, sticky after wet with saliva Flavor—Mostly neutral, bitter, cardboard, chalky, earthy

Sample: Fermented Color—Khaki, dark beige Aroma—Toasted flour, slightly sour Texture—Chalky, sticky Flavor—Sweet, chalky, bready, earthy, fruity, (banana)

Overall, cardboard, earthy notes were converted to bready, fruity, chalky, and sour notes after fermentation.

### Example 22

Red Bean Powder—90 g/L 7 Day

Two 1 L baffled DeLong Erlenmeyer flasks were filled with 0.500 L of a medium consisting of 90 g/l of red bean. The flasks were covered with a stainless-steel cap and sterilized in an autoclave on a liquid cycle that held the flasks at 120-123° C. for 1.5 hour. The flasks were carefully transferred to a clean HEPA laminar flow hood where they cooled for 4 hours and were inoculated with 10% culture of Lentinula edodes grown on medium consisting of 25 g/L glucose, 5 g/L yeast extract and 1 g/l of lecithin emulsion in oil. These flasks were placed on a shaker table at 150 rpm with a swing radius of 1″ at room temperature and incubated for 7 days. Samples were examined under microscope at the end of 7 days fermentation. A microscope check was done to ensure the presence of mycelium (mycelial pellets were visible by the naked eye) and the culture was plated on LB media to ascertain the extent of any bacterial contamination and none was observed. These cultures were dried and organoleptic tasting was conducted to determine how it differed from raw control.

Sample: Raw Color—Light sandy brown Aroma—Sweet legume, earthy, dirt Flavor—Sweet grain, chocolate, bitter, toasted grain, burnt hay, earthy

Sample: Fermented Color—Burnt red Aroma—Sour, earthy, dirt, brown spice Flavor-Caramelized, malty, earthy, dirt, astringent

Overall, sweet toasted grain, chocolate, earthy, and burnt hay notes were converted to caramelized, malty, earthy, and astringent notes after fermentation.

Red Bean—70 g/L 7 Day

Two 1 L baffled DeLong Erlenmeyer flasks were filled with 0.500 L of a medium consisting of 70 g/l of red bean. The flasks were covered with a stainless-steel cap and sterilized in an autoclave on a liquid cycle that held the flasks at 120-123° C. for 1.5 hour. The flasks were carefully transferred to a clean HEPA laminar flow hood where they cooled for 4 hours and were inoculated with 10% culture of Lentinula edodes grown on medium consisting of 25 g/L glucose, 5 g/L yeast extract and 1 g/l of lecithin emulsion in oil. These flasks were placed on a shaker table at 150 rpm with a swing radius of 1″ at room temperature and incubated for 7 days. Samples were examined under microscope at the end of 7 days fermentation. A microscope check was done to ensure the presence of mycelium (mycelial pellets were visible by the naked eye) and the culture was plated on LB media to ascertain the extent of any bacterial contamination and none was observed. These cultures were dried and organoleptic tasting was conducted to determine how it differed from raw control.

Concentration was reduced from 90 g/L to 70 g/L to help produce a more liquified product. 90 g/L was too thick and seemed to harden to the flask wall.

Red Bean—45 g/L 7 Day

Two 1 L baffled DeLong Erlenmeyer flasks were filled with 0.500 L of a medium consisting of 45 g/l of red bean. The flasks were covered with a stainless-steel cap and sterilized in an autoclave on a liquid cycle that held the flasks at 120-123° C. for 1.5 hour. The flasks were carefully transferred to a clean HEPA laminar flow hood where they cooled for 4 hours and were inoculated with 10% culture of Lentinula edodes grown on medium consisting of 25 g/L glucose, 5 g/L yeast extract and 1 g/l of lecithin emulsion in oil. These flasks were placed on a shaker table at 150 rpm with a swing radius of 1″ at room temperature and incubated for 7 days. Samples were examined under microscope at the end of 7 days fermentation. A microscope check was done to ensure the presence of mycelium (mycelial pellets were visible by the naked eye) and the culture was plated on LB media to ascertain the extent of any bacterial contamination and none was observed. These cultures were dried and organoleptic tasting was conducted to determine how it differed from raw control.

Concentration was reduced from 90 g/L to 45 g/L to help produce a more liquified product. 90 g/L was thick and seemed to harden to the flask wall.

### Example 23: “Bulking Combinations”

The following media were tested with different organisms as shown in the Examples above. These media recipes were created in 7 L Eppendorf biofermenters with a working volume of 4 L. The fermenters were sterilized in an autoclave at 120-123° C. for 2+ hours. The fermenters were then transferred to the pilot fermentation lab to be cooled for 2+ hours. Once cooled, they were individually inoculated at 10% of five different strains: Lentinula edodes, Cordyceps sinensis, Morchella esculenta, Cyclocybe aegrita and Pleurotus salmoneostramineus. These inoculums were grown in flask with a medium consisting of 25 g/L glucose, 5 g/L yeast extract, and 1 g/L of lecithin emulsion in oil as prepared in example 22. The biofermenters were allowed to ferment for 48 hours before the culture was pasteurized and harvested. A microscope check was done to ensure the presence of mycelium (mycelial pellets were visible by the naked eye) and the culture was plated on LB media to ascertain the extent of any bacterial contamination and none was observed. These fermented materials were dried and organoleptic tasting was conducted to determine how it differed from raw control. Not all combinations of strains and medium were selected for these studies.

TABLE 8

| Component | Medium 1 | Medium 2 | Medium 3 | Medium 4 |
|---|---|---|---|---|
| Pea protein 72% protein | 22.5 | 22.5 | 45 | 22.5 |
| (g/L) | | | | |
| Chickpea flour (g/L) | 22.5 | 45 | 22.5 | 22.5 |
| Corn Gluten Meal (g/L) | 45 | 22.5 | 22.5 | 22.5 |
| Rice Bran Fiber (g/ml) | 0 | 0 | 0 | 22.5 |

TABLE 9

| Component | Medium 1 | Medium 2 | Medium 3 | Medium 4 |
|---|---|---|---|---|
| Lentinula | Color: Light yellow | — | — | Color: light yellow |
| edodes | brown | | | brown |
| | Aroma: low aroma, | | | Aroma: low aroma, |
| | cardboard, corn, slight | | | cardboard, slightly |
| | grain | | | more aromatic than |
| | Flavor: cardboard, sour, | | | Shavano 1, grain. |
| | bitter, fermented, | | | Flavor: sweet, slight |
| | mushroom | | | bitter, malty, sweet |
| | Texture: gritty | | | grain, chalky. Overall |
| | | | | low-moderate flavor |
| | | | | profile. |
| | | | | Texture: chalky |
| Cordyceps | Color: Maize yellow | — | — | — |
| sinensis | Aroma: musty, moldy, | | | |
| | green | | | |
| | Flavor: sour, musty, | | | |
| | chalky, green fungal, | | | |
| | mushroom, bitter, | | | |
| | astringent. Strong flavor | | | |
| | intensity. | | | |
| | Texture: gritty, sandy | | | |
| Morchella | Color: Maize yellow | Color: Light brown | Color: Light brown | — |
| esculenta | Aroma: slight sour, corn, | Aroma: toasted, | Aroma: musty, | |
| | wet cardboard | slight fungal | fungal | |
| | Flavor: sour, burnt, | Flavor: moderate | Flavor: toasted | |
| | bitter, cardboard, corn | sweet, caramelized, | grain, mushroom, | |
| | (slight). Moderate flavor | slight sour, low | sour, slight sweet, | |
| | intensity. | fungal note, chalky, | fermented, fungal, | |
| | Texture: gritty, chalky | slight astringency. | musty, chalky. | |
| | aftertaste | Overall low- | Moderate flavor | |
| | | moderate flavor | intensity | |
| | | intensity. | Texture: chalky, | |
| | | Texture: chalky | gritty | |
| Cyclocybe | Color: Maize yellow | — | — | — |
| aegrita | Aroma: low aroma, | | | |
| | sweet grain, corn | | | |
| | Flavor: sweet, sour, | | | |
| | slight fungal, slight corn | | | |
| | at backend, dirty, | | | |
| | astringent. Low- | | | |
| | moderate flavor | | | |
| | intensity, after L. edodes | | | |
| | recipe 4 and similar to | | | |
| | L. edodes recipe 1. | | | |
| | Texture: gritty, | | | |
| | toothpack | | | |
| Pleurotus | Color: light yellow | — | — | Color: light yellow |
| salmoneo | brown | | | brown |
| stramineus | Aroma: sour, fungal, | | | Aroma: sour, |
| | floral | | | fermented, cardboard, |
| | Flavor: sweet, chalky, | | | barnyard |
| | slight sour, toasted | | | Flavor: caramelized |
| | marshmallow, fungal | | | sweet, malty, toasted |
| | (moderate), bitter. | | | grain, chalky, |
| | Dynamic flavor profile, | | | cardboard, slight |
| | changing from | | | bitter, slight fungal. |
| | beginning to end. | | | Overall low flavor |
| | Overall moderate flavor | | | intensity. Lowest yet. |
| | intensity. | | | Texture: gritty |
| | Texture: chalky | | | |

SUMMARY: P. salmoneostramineus Recipe 4 were most preferred due to low flavor intensity followed by L. edodes Recipe 4.

M. esculenta Recipe 3 has more flavor intensity and bitterness than M. esculenta Recipe 1 and 2. Therefore M. esculenta Recipe 3 was least preferred compared to Recipes 1 and 2.

C. sinensis Recipe 1 has highest flavor intensity and highest fungal/musty notes, causing it to be least preferable of all samples.

Top 3 strains: M. esculenta, P. salmoneostramineus, and L. edodes.

### Example 24: Different Strains with Corn Gluten Meal

Two 1 L baffled DeLong Erlenmeyer flasks were filled with 0.500 L of a medium consisting of 90 g/l of corn gluten meal (Ingredion, Prairie Gold® 60% (protein) Corn Gluten Meal 138930. The flasks were covered with a stainless-steel cap and sterilized in an autoclave on a liquid cycle that held the flasks at 120-123° C. for 1.5 hour. The flasks were carefully transferred to a clean HEPA laminar flow hood where they cooled for 4 hours and were inoculated with 10% culture of Lentinula edodes, Morchella esculenta, or Pleurotus salmoneostramineus, grown on medium consisting of 25 g/L glucose, 5 g/L yeast extract and 1 g/l of lecithin emulsion in oil. These flasks were placed on a shaker table at 150 rpm with a swing radius of 1″ at room temperature and incubated for 7 days. Samples were examined under microscope at the end of 7 days fermentation. A microscope check was done to ensure the presence of mycelium (mycelial pellets were visible by the naked eye) and the culture was plated on LB media to ascertain the extent of any bacterial contamination and none was observed. These cultures were dried, and organoleptic tasting was conducted to determine how it differed from raw corn gluten meal. Results shown in Table 10.

TABLE 10

| | |
|---|---|
| Sample: Control | Color: mustard yellow |
| (90 g/L Corn | Aroma: sulfur (strong), corn |
| Gluten Meal) | Flavor: chalky, corn, slight sweet, slight sour, |
| | sulfur (moderate-high), slight metallic, dirt/earthy |
| | Texture: gritty, sandy |
| Sample: | Color: light tan, sawdust |
| L. edodes | Aroma: mild, slight woody, slight sour, |
| 7 day flasks | reduced sulfur aroma |
| | Flavor: sour (mid-high), woody/bark, bread, |
| | warming, slight corn, refined grain, bitter |
| | Texture: gritty |
| Sample: P. | Color: Pale Khaki |
| salmoneostramineus | Aroma: dried medicinal roots/vitamins/woody |
| 7 day flasks | Flavor: sweet (mid-high intensity), burnt |
| | marshmallow, slight sour, woody, medicinal roots, |
| | vitamin, bitter at backend, metallic/vitamin linger |
| | Texture: Chalky |
| Sample: | Color: Pale mustard yellow |
| M. esculenta | Aroma: sour, corn (predominant), slight sulfur, |
| 7 day flasks | earthy |
| | Flavor: sour (low), burnt, bitter, corn, sulfur |
| | (low) |
| | Texture: Gritty |

M. esculenta was most similar to control, with slight decrease of sour and sulfur. L. edodes was noticeably sour, but reduced sulfur. P. salmoneostramineus was preferred due to higher sweetness and elimination of corn flavor.

### Example 25

A chocolate-flavored paste was prepared as follows. The mixture was milled into a smooth, spreadable paste. The paste had a water activity of less than about 0.86. The taste was rich and creamy. See Table 11.

TABLE 11

| Ingredient | Weight % | grams |
|---|---|---|
| Hazelnuts | 36.56% | 140.00 |
| Bulking dried powder (prepared as in Example 24) | 14.36% | 55.00 |
| Sugar, granulated | 16.98% | 65.00 |
| Crisco | 6.53% | 25.00 |
| Dutch Processed Cocoa Powder | 2.73% | 10.45 |
| Melted Milk Chocolate (cadbury) | 21.94% | 84.00 |
| Salt | 0.00% | |
| Stevia, 97 | 0.03% | 0.10 |
| Lecithin | 0.88% | 3.36 |

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A method to prepare an improved composition comprising at least one low-quality protein, comprising the steps of:
providing an aqueous media comprising at least one low-quality protein, wherein the media comprises at least 10 g/L low-quality protein;
inoculating the medium with a filamentous fungal culture, wherein the fungal culture comprises Lentinula spp., Pleurotus spp., or Morchella spp., and
culturing the medium in submerged fungal culture to produce a myceliated low-quality protein composition;
wherein the myceliated low-quality protein composition has reduced one or more undesirable aroma and/or reduced one or more undesirable tastes, compared to a low-quality protein composition that is not myceliated.

2. The method of claim 1, wherein the composition is a bulking ingredient composition or a protein concentrate composition.

3. The method of claim 1, wherein the myceliated composition has decreased bitter, beany, and/or earthy tastes and decreased sulfur, and/or earthy aromas.

4. The method of claim 1, wherein when the filamentous fungus culture is selected from the group consisting of Lentinula edodes, Pleurotus ostreatus, Pleurotus salmoneostramineus (Pleurotus djamor) and Morchella esculenta.

5. The method of claim 1, wherein the composition comprises rice bran fiber, chicory root, grapeseed, or spent beer grains.

6. The method of claim 1, wherein the low-quality protein is a bean or corn gluten meal and in the form of a powder.

7. The method of claim 1, wherein the bean is red bean or fava bean.

8. The method of claim 1, wherein the aqueous media additionally comprises a high protein composition from a plant source comprising pea, rice, chickpea, hemp, oat, soybean, or combinations thereof.

9. The method of claim 1, wherein the aqueous media comprises chickpea flour, pea protein, and corn gluten meal.

10. The method of claim 9, wherein the filamentous fungus is selected from the group of L. edodes, M. esculenta, and P. salmoneostramineus.

11. The method of claim 1, wherein the aqueous media comprises between 10 g/L protein and 75 g/L protein.

12. The method of claim 1, wherein the method further comprises the step of drying the myceliated low-quality protein composition.

13. The method of claim 1, wherein the pH of the fungal culture has a change of less than 0.3 pH units during the culturing step.

14. A myceliated low-quality protein composition made by the method of claim 1.

15. A composition comprising a myceliated low-quality protein composition, wherein the myceliated low-quality protein composition is at least 20% (w/w) protein on a dry weight basis, wherein myceliated low-quality protein composition is myceliated by filamentous fungal culture comprising Lentinula spp., Pleurotus spp., or Morchella spp. in a media comprising at least 20 g/L protein, and wherein the myceliated low-quality protein composition has decreased undesirable aromas and/or decreased undesirable tastes, compared to a low-quality protein composition that is not myceliated.

16. The composition of claim 15, wherein the composition is a bulking ingredient composition or a protein concentrate composition.

17. The composition of claim 15, wherein the myceliated composition has myceliated composition has decreased bitter, beany, and/or earthy tastes and decreased sulfur, and/or earthy aromas.

18. The composition of claim 15, wherein when the filamentous fungus culture is selected from the group consisting of Lentinula edodes, Pleurotus ostreatus, Pleurotus salmoneostramineus (Pleurotus djamor) and Morchella esculenta.

19. The composition of claim 15, wherein the media comprises rice bran fiber, chicory root, grapeseed, or spent beer grains.

20. The composition of claim 15, wherein the low-quality protein is a bean or corn gluten meal.

21. The composition of claim 15, wherein the bean is red bean or fava bean.

22. The composition of claim 15, wherein the media additionally comprises a high protein composition from a plant source comprising pea, rice, chickpea or combinations thereof.

23. The composition of claim 15, wherein the aqueous media comprises chickpea flour, pea protein, and corn gluten meal.

24. The composition of claim 15, wherein the filamentous fungus is selected from the group of L. edodes, M esculenta, and P. salmoneostramineus.

25. A food composition comprising the composition of claim 15 or claim 14.

26. The food composition of claim 26, wherein the food composition is an extruded food composition.

27. The food composition of claim 26, wherein the food composition is selected from the group consisting of spreads, pastes, prewhipped toppings, custards, coatings, nut butters, frostings, cream filings, confectionery fillings, dairy alternative products, beverages and beverage bases, extruded and extruded/puffed products, meat imitations and extenders, baked goods and baking mixes, granola products, bar products, smoothies and juices, and soups and soup bases.
